(19) United States
(12) Patent Application Publication
(10) Pub. No.: US 2022/0257886 A1
(43) Pub. Date: Aug. 18, 2022

(54) MONITORING DEVICE, MONITORING SYSTEM, AND STATE PARAMETER ACQUISITION METHOD

(71) Applicant: BOE TECHNOLOGY GROUP CO., LTD., Beijing (CN)

(72) Inventors: Haiyun Cui, Beijing (CN); Zigang Liu, Beijing (CN); Yang Peng, Beijing (CN)

(73) Assignee: BOE TECHNOLOGY GROUP CO., LTD., Beijing (CN)

(21) Appl. No.: 17/628,825

(22) PCT Filed: Apr. 23, 2021
(86) PCT No.: PCT/CN2021/089325

§ 371 Date: Jan. 20, 2022

(30) Foreign Application Priority Data
Apr. 30, 2020 (CN) 202010366588.8

(51) Int. Cl. A61M 16/00; A61B 5/00

(52) U.S. Cl. CPC A61M 16/0003; A61B 5/0002; A61M 16/0051; A61M 2205/3368; A61M 2205/33; A61M 2205/3331; A61M 2016/0033

(57) ABSTRACT

A monitoring device (100), a monitoring system (200) and a method for acquiring a state parameter. The monitoring device (100) includes: a sensor interface (101), a controller (102), a switch element (103) and a monitoring element (104); the sensor interface (101) is electrically connected to the monitoring element (104) and the switch element (103), and the sensor interface (101) is configured to be accessed by a sensor (203); the controller (102) is connected to the sensor interface (101) via the monitoring element (104) and the switch element (103) that are connected in parallel; the monitoring element (104) is configured to, when the sensor interface (101) is monitored to be accessed by a sensor (203), transmit a monitoring information to the controller (102); and the controller (102) is configured to, control an on-off state of a switch in the switch element (103), to select the specified sensor (203) to be powered.

### CROSS REFERENCE TO RELEVANT APPLICATIONS

The present application claims the priority of the Chinese patent application filed on Apr. 30, 2020 before the Chinese Patent Office with the application number of 202010366588.8 and the title of “MONITORING DEVICE, MONITORING SYSTEM, AND STATE PARAMETER ACQUISITION METHOD”, which is incorporated herein in its entirety by reference.

### TECHNICAL FIELD

The present disclosure relates to the technical field of information monitoring, and particularly relates to a monitoring device, a monitoring system and a state parameter acquisition method.

### BACKGROUND

Respiration devices (such as a breathing machine, an oxygenerator, a diving mask and a gas mask, etc.) operate by, in autonomous respiration, performing the inhalation action to generate a pleural-cavity negative pressure, whereby the lung is passively expanded to generate a negative pressure in the alveolus and the gas ducts, thereby forming a pressure difference between the gas duct ports and the alveolus to complete the inhalation, and, after the inhalation, retracting elastically the thoracic cage and the lung, to generate an opposite pressure difference to complete the expiration.

### SUMMARY

The present disclosure provides a monitoring device, a monitoring system and a method for acquiring a state parameter.

The present disclosure discloses a monitoring device, wherein the monitoring device comprises: a sensor interface, a controller, a switch element and a monitoring element;

the sensor interface is electrically connected to the monitoring element and the switch element, and the sensor interface is configured to be accessed by a sensor;

the controller is connected to the sensor interface via the monitoring element and the switch element that are connected in parallel;

the monitoring element is configured to, transmit a monitoring information to the controller when the sensor interface is monitored to be accessed by a sensor; and

the controller is configured to, according to the monitoring information sent by the monitoring element, control an on-off state of a switch in the switch element, to select to a specified sensor to be powered on among the accessed sensors, and obtain a sensing information of the specified sensor.

Optionally, the controller is further configured to, in an operating process of the specified sensor, receive a current or voltage signal outputted by the specified sensor, and according to the current or voltage signal and a physical-parameter conversion formula corresponding to the specified sensor, obtain a state parameter corresponding to the sensing information by calculation.

Optionally, the monitoring device further comprises: a communication element;

the communication element is connected to the controller;

the controller is further configured to output the state parameter to the communication element; and

the communication element is configured to receive the state parameter sent by the controller, and send the state parameter to a receiving device.

Optionally, the communication element is any one of a wireless-fidelity module, a Bluetooth module, a fourth-generation-mobile-communication-technology module and a fifth-generation-mobile-communication-system module.

Optionally, the monitoring device further comprises: a power-supply interface; and

the power-supply interface is electrically connected to a pin of the sensor interface, and the power-supply interface is configured to be connected to a power supply, to provide electric energy to operation of the monitoring device.

Optionally, the monitoring element is a current monitoring chip; and

the current monitoring chip is configured to, when the sensor interface is accessed by the sensor and the power-supply interface switches on the power supply, transmit a current signal flowing through the current monitoring chip to the controller.

Optionally, the controller is further configured to, according to a current signal sent by the current monitoring chip, identify an accessing state of the sensor interface.

Optionally, the monitoring device further comprises: a locating element; and

the locating element is connected to the communication element, and the locating element is configured to locate a position information of the monitoring device, and send the position information to the receiving device.

Optionally, the sensor interface and the switch element comprise quantity of switches comprised in the sensor interface is equal to the quantity of switches comprised in the switch element, and each of the sensor interfaces is electrically connected to one of the switches.

Optionally, each of the sensor interfaces corresponds to one type of sensor.

Optionally, the sensing information comprises a gas-flow-state information.

The present disclosure discloses a monitoring system, wherein the monitoring system comprises: a breathing device, a sensor and the monitoring device according to any one of the above items.

Optionally, the sensor accesses the sensor interface of the monitoring device, and the monitoring device is provided inside of a gas-flow channel of the breathing device.

Optionally, the monitoring system further comprises: a receiving device; and

the receiving device is configured to receive state parameter inside of a gas-flow channel of the breathing device that is sent by the monitoring device, and the position information sent by the monitoring device.

Optionally, the receiving device is provided with an alarming element; and

the alarming element is configured to, when the state parameter triggers a preset alarming condition, generate an alarming information, and send the alarming information to a target device.

Optionally, the sensor comprises: one or more of a pressure sensor, a flow sensor, a temperature sensor, a humidity sensor, an oxygen-concentration sensor, a position sensor and a carbon-dioxide-concentration sensor.

The present disclosure discloses a method for acquiring a state parameter, wherein the method is applied to the monitoring system according to any one of the above items, and the method comprises:

according to a received current signal of the monitoring element, determining whether the sensor interface is accessed by a sensor of a specified type;

when it is determined that the sensor interface has been accessed by a sensor of the specified type, controlling, by the controller, a switch in the switch element that corresponds to the sensor interface to be conductive; and

acquiring, by the sensor of the specified type, a state parameter inside of a gas-flow channel of the breathing device.

Optionally, after the step of acquiring, by the sensor of the specified type, the state parameter inside of the gas-flow channel of the breathing device, the method further comprises:

sending the state parameter to a receiving device; and

when the state parameter triggers a preset alarming condition, generating, by the receiving device, an alarming information, and sending, by the receiving device, the alarming information to a target device.

The present disclosure further discloses a calculating and processing device, wherein the calculating and processing device comprises:

a memory storing a computer-readable code; and

one or more processors, wherein when the computer-readable code is executed by the one or more processors, the calculating and processing device implements the method for acquiring a state parameter stated above.

The present disclosure further discloses a computer program, wherein the computer program comprises a computer-readable code, and when the computer-readable code is executed in a calculating and processing device, the computer-readable code causes the calculating and processing device to implement the method for acquiring a state parameter stated above.

The present disclosure further discloses a computer-readable medium, wherein the computer-readable medium stores the computer program stated above.

The above description is merely a summary of the technical solutions of the present disclosure. In order to more clearly know the elements of the present disclosure to enable the implementation according to the contents of the description, and in order to make the above and other purposes, features and advantages of the present disclosure more apparent and understandable, the particular embodiments of the present disclosure are provided below.

### BRIEF DESCRIPTION OF THE DRAWINGS

In order to more clearly illustrate the technical solutions of the embodiments of the present disclosure or the related art, the figures that are required to describe the embodiments or the related art will be briefly introduced below. Apparently, the figures that are described below are merely embodiments of the present disclosure, and a person skilled in the art can obtain other figures according to these figures without paying creative work.

FIG. 1 shows a schematic structural diagram of the monitoring device according to an embodiment of the present disclosure;

FIG. 2 shows a schematic structural diagram of the monitoring device according to another embodiment of the present disclosure;

FIG. 3 shows a schematic diagram of the operating state of the monitoring device according to an embodiment of the present disclosure;

FIG. 4 shows a schematic structural diagram of the monitoring system according to an embodiment of the present disclosure;

FIG. 5 shows a schematic structural diagram of the monitoring system according to another embodiment of the present disclosure;

FIG. 6 shows a flow chart of the steps of the method for acquiring a state parameter according to an embodiment of the present disclosure;

FIG. 7 schematically shows a block diagram of a calculating and processing device for implementing the method according to the present disclosure; and

FIG. 8 schematically shows a storage unit for maintaining or carrying a program code for implementing the method according to the present disclosure.

### DETAILED DESCRIPTION OF THE EMBODIMENTS

In order to make the above purposes, features and advantages of the present disclosure more apparent and understandable, the present disclosure will be described in further detail below with reference to the drawings and the particular embodiments.

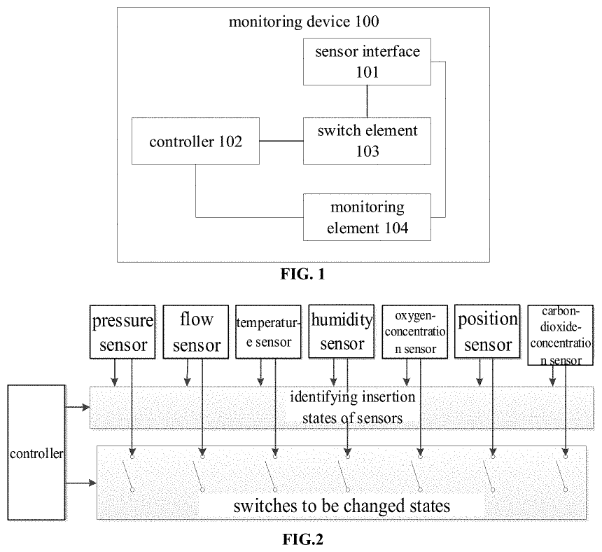

As referred to FIG. 1, FIG. 1 shows a schematic structural diagram of the monitoring device according to an embodiment of the present disclosure. As shown in FIG. 1, the monitoring device 100 comprises: a sensor interface 101, a controller 102, a switch element 103 and a monitoring element 104; and

the sensor interface 101 is electrically connected to the monitoring element 104 and the switch element 103, and the sensor interface 101 may be configured to be accessed by a sensor.

In the present embodiment, the monitoring device 100 may comprise a mainframe, a plurality of sensor interfaces 101 are disposed at the external surface of the mainframe, and the controller 102, the switch element 103 and the monitoring element 104 are disposed at the inside of the mainframe.

Each of the sensor interfaces 101 corresponds to one type of sensor, and each of the sensor interfaces 101 is configured to be accessed by the corresponding type of sensor. For example, the sensor interfaces may comprise a sensor interface 1, a sensor interface 2, a sensor interface 3 and a sensor interface 4, wherein the sensor interface 1 corresponds to a pressure sensor, the sensor interface 2 corresponds to a humidity sensor, a sensor interface 3 corresponds to a temperature sensor, and the sensor interface 4 corresponds to an oxygen-concentration sensor. Accordingly, the sensor interface 1 is configured to be accessed by a pressure sensor, the sensor interface 2 is configured to be accessed by a humidity sensor, the sensor interface 3 is configured to be accessed by a temperature sensor, and the sensor interface 4 is configured to be accessed by an oxygen-concentration sensor.

It can be understood that the above example is merely an example that is provided for a better comprehension of the technical solution of the embodiments of the present disclosure, and should not be deemed a sole limitation on the embodiments of the present disclosure.

The controller 102 may be connected to the sensor interfaces 101 via the monitoring element 104 and the switch element 103 that are connected in parallel.

One or more switches are provided in the switch element 103. Particularly, the quantity of the switches in the switch element 103 is equal to the quantity of the sensor interfaces 101, and each of the sensor interfaces 101 is electrically connected to one of the switches. The on-off states of the switches in the switch element 103 are controlled by the controller. Particularly, according to the accessing state of the sensor, the plurality of switches are selected to be turned on by the I/O port of the controller, and the on-off states of the switches are controlled by using the high/low levels of the I/O port. For example, if a switch is turned on to be conductive by a high-level voltage and turned off by a low-level voltage, then the controller may send a high-level voltage via the I/O port to turn on the switch, and the controller may also send a low-level voltage via the I/O port to turn off the switch, thereby realizing controlling the on-ff state of the switch.

The monitoring element 104 may be configured to, transmit a monitoring information to the controller, when the sensor interface is monitored to be accessed by a sensor. The embodiment takes the monitoring element as a current monitoring chip as an example, as shown in FIG. 2, each of the sensor interfaces is connected to one current monitoring chip in series. When a sensor accesses to each of the sensor interfaces, after the power supply is switched on, a current flow through the current monitoring chip, and feeds back the current signal to the controlling unit. The controlling unit identifies the current signal according to its channels, whereby it may determine the type of the accessed sensor, that is, which of the sensor interfaces is accessed by a sensor.

The controller 102 may, according to the monitoring information sent by the monitoring element 104, control an on-off state of a switch in the switch element, to select a specified sensor to be powered on among the accessed sensors, and obtain a sensing information of the specified sensor.

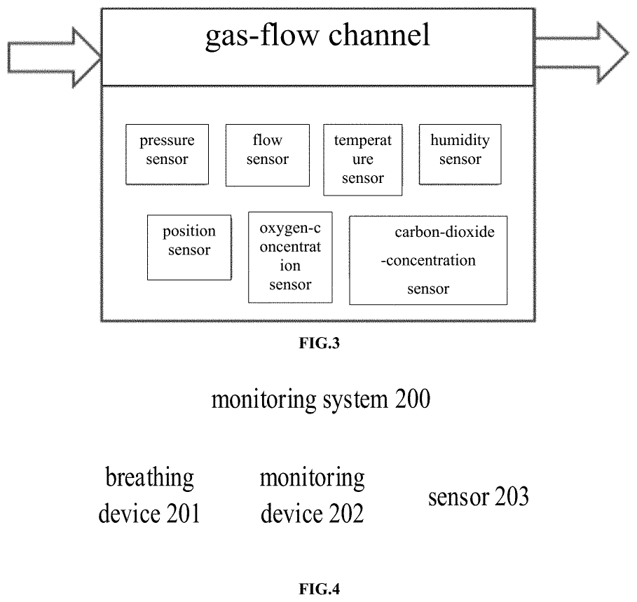

In the present embodiment, the sensing information may be a gas-flow-state information. When the device is used to monitor the gas-flow state inside of the gas-flow channel of a breathing device, a sensor of a specified type may be accessed by the specified sensor interface according to the demand of the monitoring, to monitor the gas-flow state inside of the gas-flow channel of the breathing device. As shown in FIG. 3, in the usage of the monitoring device, the monitoring device may be installed inside of the gas-flow channel of the breathing device, to monitor the gas-flow state inside of the gas-flow channel. For example, when it is required to monitor the humidity inside of the gas-flow channel of the breathing device, the monitoring device may be accessed by a humidity sensor, and the monitoring device may be disposed inside of the gas-flow channel, to monitor the gas-flow humidity inside of the gas-flow channel. Certainly, the method is not limited thereto, and, in particular implementations, when it is required to monitor multiple gas-flow states inside of the gas-flow channel, the corresponding sensors may be installed according to the demands, which may be particularly configured according to the service demands.

In a particular implementation of the present disclosure, the controller 102 may further, in the operating process of the specified sensor, receive a current or voltage signal outputted by the specified sensor, and according to the current or voltage signal and a physical-parameter conversion formula corresponding to the specified sensor, obtain a state parameter corresponding to the sensing information by calculation.

In the present embodiment, after the controller 102 has acquired the state parameter, the state parameter may be outputted to an external device. Particularly, the monitoring device 100 may further comprise a communication element (not shown in the FIGS.), the communication element may be connected to the controller 102, the controller 102 may output the state parameter to the communication element, and the communication element further sends the state parameter to a receiving device (such as a mobile phone and a computer).

The communication element may be a wireless-fidelity (WiFi) module, a Bluetooth module, a fourth-generation-mobile-communication-technology (4G)/fifth-generation-mobile-communication-system (5G) module and so on, which may be particularly configured according to the service demands, and is not limited in the present embodiment. The WiFi module belongs to the transport layer of the internet of things, is an embedded-type module that may convert a serial port or TTL electrical level to satisfy the WiFi wireless-network communication standards, and is an important hardware component of the monitoring device. The Bluetooth module is a PCBA board integrated with the function of Bluetooth, and by embedding the Bluetooth module into the monitoring device, short-distance wireless communication of the monitoring device may be realized. The 4G module and the 5G module are generic terms for products in which the hardware is loaded to the specified frequency bands and the software supports the LTE protocol in the 4G and 5G standards respectively, and by installing the 4G module or 5G module in the monitoring device, data transmission of a high speed, a long distance and a large capacity may be realized.

In another particular implementation of the present disclosure, the monitoring device 100 may further comprise a power-supply interface (not shown in FIGS.). The power-supply interface may be electrically connected to a pin of the sensor interface. The power-supply interface may be electrically connected to the power supply when the monitoring device 100 is operating, to provide electric energy to operation of the monitoring device.

Certainly, the controller 102 may, when the power-supply interface is connected to the power supply, according to a current signal sent by the current monitoring chip, identify the accessing state of the sensor interface. The accessing state may particularly be which of the sensor interfaces is accessed by a sensor; in other words, that corresponds to the above-described example of the identification of the current monitoring chip.

In another particular implementation of the present disclosure, the monitoring device 100 may further comprise a locating element (not shown). The locating element may be connected to the communication element. The locating element may locate the position information of the monitoring device 100 in real time, and send the position information to the receiving device. Regarding diving devotees or users working in particular environments (such as workers in mines, and users in places where it is required to wear a gas mask), by accessing the monitoring device into the gas path, the position of the user and the state of the respiratory gas flow may be monitored in real time. When hypoxia, asphyxia and so on happen, rescue workers may be alerted to rescue in time, and the rescue workers may be provided with the services of locating and navigation. Moreover, regarding physically inconvenienced users such as the elderly, in the usage of a breathing machine or an oxygenerator, by accessing the device into the gas path, the state of the respiratory gas flow of the user may be monitored in real time, and, when poor comfortableness, hypoxia, asphyxia and so on happen, the guardian may be alerted in time, to ensure the safety of the user.

The monitoring device according to the embodiments of the present disclosure may comprise: a sensor interface, a controller, a switch element and a monitoring element, wherein the sensor interface is electrically connected to the monitoring element and the switch element, and is configured to be accessed by a sensor; the controller is connected to the sensor interface via the monitoring element and the switch element that are connected in parallel; the monitoring element is configured to, when the sensor interface is monitored to be accessed by a sensor, transmit a monitoring information to the controller; and the controller is configured to, according to the monitoring information sent by the monitoring element, control an on-off state of a switch in the switch element, to select a specified sensor to be powered on among the accessed sensors, and obtain a sensing information of the specified sensor. The monitoring device according to the embodiments of the present disclosure may monitor the gas-flow state inside of the breathing device in real time, and, when poor comfortableness, hypoxia, asphyxia and so on happen, alert the guardian in time, to ensure the safety of the user.

As referred to FIG. 4, FIG. 4 shows a schematic structural diagram of the monitoring system according to an embodiment of the present disclosure. As shown in FIG. 4, the monitoring system 200 may comprise: a breathing device 201, a monitoring device 202 and a sensor 203, and the monitoring device 202 is the monitoring device described in the above embodiments.

In the process of usage, the sensor 203 may access the sensor interface of the monitoring device 202, and the monitoring device 202 is disposed inside of a gas-flow channel of the breathing device 201, to monitor the gas-flow state inside of the gas-flow channel of the breathing device 201.

In the present embodiment, the sensor 203 may comprise: one or more of sensors such as a pressure sensor, a flow sensor, a temperature sensor, a humidity sensor, an oxygen-concentration sensor, a position sensor and a carbon-dioxide-concentration sensor. Particularly, the corresponding sensors may be installed to the monitoring device 202 according to practical demands, which is not limited in the present embodiment.

In the present embodiment, the monitoring system 200 may further comprise a receiving device (not shown in FIGS.).

The receiving device may be a terminal device (such as a mobile phone and a computer). The receiving device may receive the state parameter inside of the gas-flow channel of the breathing device that is sent by the monitoring device 202 and the position information sent by the monitoring device 202.

The receiving device is further provided with an alarming element. The alarming element may, when the state parameter triggers a preset alarming condition, generate an alarming information, and send the alarming information to a target device. The alarming element may be a software or a hardware. When the alarming element is a hardware, when the state parameter triggers a preset alarming condition, an alarming information is generated, for example, generating an electric signal, and it is sent to a target device. For example, regarding diving devotees or users working in particular environments (such as workers in mines, and users in places where it is required to wear a gas mask), by accessing the device according to the present disclosure into the gas path, the position of the user and the state of the respiratory gas flow may be monitored in real time. The preset alarming condition may be a state parameter such as the gas-flow pressure, the gas-flow rate, the gas-flow humidity and the gas-flow temperature is not within the preset range, which may result in hypoxia, asphyxia and so on of the user. At this point, the alarming element may, according to the particular abnormal data, generate a corresponding alarming information, and send the alarming information to a target device, to alert rescue workers to rescue in time, and provide the services of locating and navigation for the rescue workers. Particularly, that may refer to FIG. 5 for detailed description.

As shown in FIG. 5, when the user is using the breathing device such as a breathing machine, an oxygenerator, a diving mask and a gas mask, the respiratory-gas-flow sensing device (i.e., the monitoring device) may monitor the gas-flow state inside of the gas-flow channel of the breathing device in real time, and send the gas-flow-state parameter that is monitored to a cloud side. The cloud side may analyze the gas-flow state, to determine whether abnormality is happened, and send the parameter indicating whether the gas-flow state is abnormal to a personal computer (PC) side and a specified application program (APP), to monitor the gas flow of the breathing device in real time. If the gas-flow state has an abnormal parameter, which indicates that the user may have conditions such as poor comfortableness, hypoxia and asphyxia, the guardian may, according to the alarming information of the abnormal parameter that is sent by the alarming element, rescue the user in time, to ensure the safety of the user.

The monitoring system according to the embodiments of the present disclosure may monitor the gas-flow state inside of the gas-flow channel of the breathing device in real time, and, when poor comfortableness, hypoxia, asphyxia and so on of the user happen, alert the guardian in time, to ensure the safety of the user.

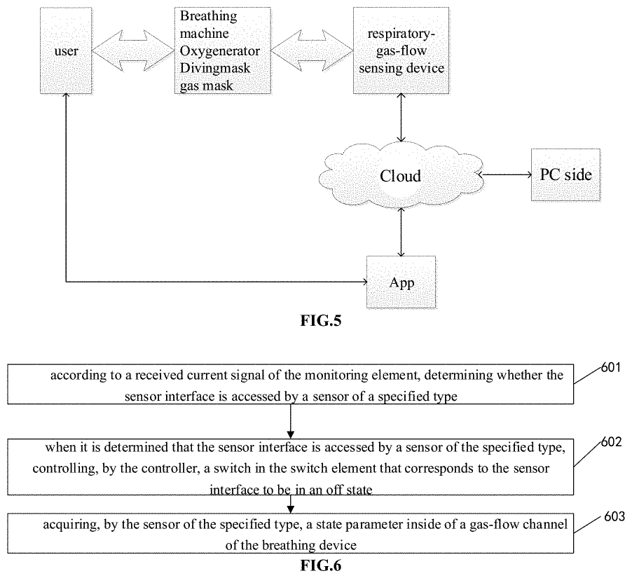

Referring to FIG. 6, FIG. 6 shows a flow chart of the steps of the method for acquiring a state parameter according to an embodiment of the present disclosure. The method for acquiring a state parameter may be applied to the above-described monitoring system, and may particularly comprise the following steps:

Step 601: according to a received current signal of the monitoring element, determining whether the sensor interface is accessed by a sensor of a specified type.

In an embodiment of the present disclosure, the sensor may be one or more of sensors such as a pressure sensor, a flow sensor, a temperature sensor, a humidity sensor, an oxygen-concentration sensor, and a carbon-dioxide-concentration sensor. Particularly, the corresponding sensors may be installed to the monitoring device according to practical demands, which is not limited in the present embodiment.

The monitoring element is disposed inside of the monitoring device. The monitoring element may monitor whether the sensor interface is accessed by a sensor of a specified type. Particularly, taking the case as an example in which the monitoring element is a current monitoring chip, as shown in FIG. 2, each of the sensor interfaces is connected in series to one current monitoring chip. When a sensor is accessed to the sensor interface, after the power supply is switched on, a current flow through the current monitoring chip, and feeds back the current signal to the controlling unit. The controlling unit identifies the current signal according to channel, whereby it may determine the type of the accessed sensor, i.e., which of the sensor interfaces is accessed by a sensor.

Certainly, the method is not limited thereto, and, in particular implementations, other modes may also be used to determine whether the sensor interface is accessed by a sensor of a specified type, which may be particularly configured according to the service demands, and is not limited in the present embodiment.

After the step of, according to the received current signal of the monitoring element, determining whether the sensor interface is accessed by a sensor of a specified type, the step 602 is executed.

Step 602: when it is determined that the sensor interface is accessed by a sensor of the specified type, controlling, by the controller, a switch in the switch element that corresponds to the sensor interface to be in an on state.

When it is determined that the sensor interface is accessed by a sensor of a specified type, the controller disposed inside of the monitoring device may be used to control the switch in the switch element that corresponds to the sensor interface to be in an on state, to realize switching-on.

After controlling the switch in the switch element that corresponds to the sensor interface to be in an on state, the step 603 is executed.

Step 603: acquiring, by the sensor of the specified type, a state parameter inside of a gas-flow channel of the breathing device.

After controlling the switch in the switch element that corresponds to the sensor interface to be in an on state, the state parameter inside of the gas-flow channel of the breathing device may be monitored by using the sensor of the specified type accessing the sensor interface. Particularly, the controller inside of the monitoring device may, in the operating process of the sensor of the specified type, receive the current signal outputted by the sensor of the specified type, and, according to the current signal and a current-parameter conversion formula corresponding to the sensor of the specified type, obtain the state parameter of the gas flow by calculation.

In a particular implementation of the present disclosure, after the step 603, the method may further comprise:

Step M1: sending the state parameter to a receiving device.

In the present embodiment, the receiving device refers to a device configured to receive the state parameter. The receiving device may be a terminal, for example a device such as a mobile phone and a computer, which may be particularly configured according to the service demands, and is not limited in the present embodiment.

The communication element is disposed inside of the monitoring device. After the step of acquiring, by the sensor of the specified type, the state parameter inside of the gas-flow channel of the breathing device, the state parameter may be sent to the receiving device by using the communication element, and, after that, the step M2 is executed.

Step M2: when the state parameter triggers a preset alarming condition, generating, by the receiving device, an alarming information, and sending, by the receiving device, the alarming information to a target device.

The preset alarming condition refers to an alarming condition that is set in advance by the service personnel. For example, in the case in which the state parameter is a gas-flow-humidity parameter, a normal humidity range may be set in advance, and when the gas-flow humidity inside of the gas-flow channel of the breathing device is monitored beyond the normal humidity range, which indicates that the gas-flow humidity triggers the alarming condition.

When the state parameter triggers a preset alarming condition, the alarming information may be generated by the receiving device, and the receiving device sends the alarming information to a target device. For example, in the usage of the breathing device by the user, when discomfort, hypoxia, asphyxia and so on happen, the alarming is raised in time, to ensure the safety of the user.

The method for acquiring a state parameter according to the embodiments of the present disclosure comprises, according to a received current signal of the monitoring element, determining whether the sensor interface is accessed by a sensor of a specified type; when it is determined that the sensor interface is accessed by a sensor of the specified type, controlling the switch in the switch element that corresponds to the sensor interface to be in an on state; and acquiring, by the sensor of the specified type, a state parameter inside of a gas-flow channel of the breathing device. The embodiments of the present disclosure may monitor the gas-flow state inside of the breathing device in real time, and, when poor comfortableness, hypoxia, asphyxia and so on happen, alert the guardian in time, to ensure the safety of the user.

Regarding the above-described process embodiments, for brevity of the description, all of them are expressed as the combination of a series of actions, but a person skilled in the art should know that the present disclosure is not limited by the sequences of the actions that are described, because, according to the present disclosure, some of the steps may have other sequences or be performed simultaneously. Secondly, a person skilled in the art should also know that all of the embodiments described in the description are preferable embodiments, and not all of the actions and the modules that they involve are required by the present disclosure.

The above-described device embodiments are merely illustrative, wherein the units that are described as separate components may or may not be physically separate, and the components that are displayed as units may or may not be physical units; in other words, they may be located at the same one location, and may also be distributed to a plurality of network units. Some or all of the modules may be selected according to the actual demands to realize the purposes of the solutions of the embodiments. A person skilled in the art can understand and implement the technical solutions without paying creative work.

Each component embodiment of the present disclosure may be implemented by hardware, or by software modules that are operated on one or more processors, or by a combination thereof. A person skilled in the art should understand that some or all of the functions of some or all of the components of the calculating and processing device according to the embodiments of the present disclosure may be implemented by using a microprocessor or a digital signal processor (DSP) in practice. The present disclosure may also be implemented as device or device programs (for example, computer programs and computer program products) for implementing part of or the whole of the method described herein. Such programs for implementing the present disclosure may be stored in a computer-readable medium, or may be in the form of one or more signals. Such signals may be downloaded from an Internet website, or provided on a carrier signal, or provided in any other forms.

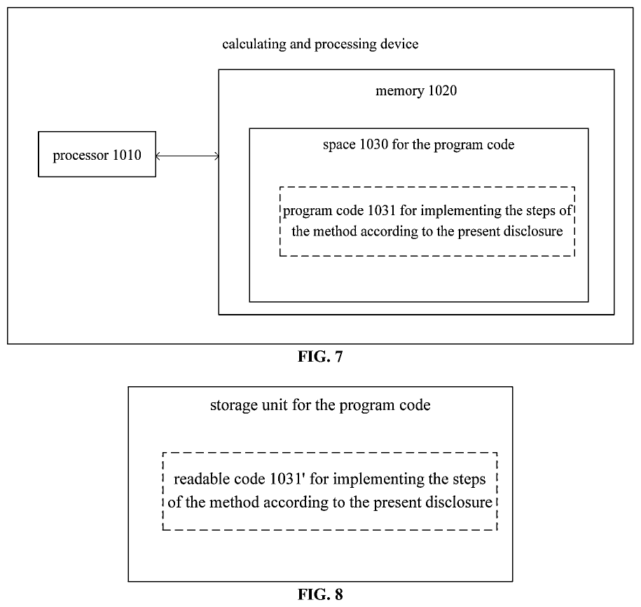

For example, FIG. 7 shows a calculating and processing device that can implement the method according to the present disclosure. The calculating and processing device traditionally comprises a processor 1010 and a computer program product or computer-readable medium in the form of a memory 1020. The memory 1020 may be electronic memories such as flash memory, EEPROM (Electrically Erasable Programmable Read Only Memory), EPROM, hard disk or ROM. The memory 1020 has the storage space 1030 of the program code 1031 for implementing any steps of the above method. For example, the storage space 1030 for program code may contain program codes 1031 for individually implementing each of the steps of the above method. Those program codes may be read from one or more computer program products or be written into the one or more computer program products. Those computer program products include program code carriers such as a hard disk, a compact disk (CD), a memory card or a floppy disk. Such computer program products are usually portable or fixed storage units as shown in FIG. 8. The storage unit may have storage segments or storage spaces with similar arrangement to the memory 1020 of the calculating and processing device in FIG. 7. The program codes may, for example, be compressed in a suitable form. Generally, the storage unit contains a computer-readable code 1031’, which can be read by a processor like 1010. When those codes are executed by the calculating and processing device, the codes cause the calculating and processing device to implement each of the steps of the method described above.

The embodiments of the description are described in the mode of progression, each of the embodiments emphatically describes the differences from the other embodiments, and the same or similar parts of the embodiments may refer to each other.

Finally, it should also be noted that, in the present text, relation terms such as first and second are merely intended to distinguish one entity or operation from another entity or operation, and that does not necessarily require or imply that those entities or operations have therebetween any such actual relation or order. Furthermore, the terms “include”, “comprise” or any variants thereof are intended to cover non-exclusive inclusions, so that processes, methods, articles or devices that include a series of elements do not only include those elements, but also include other elements that are not explicitly listed, or include the elements that are inherent to such processes, methods, articles or devices. Unless further limitation is set forth, an element defined by the wording “comprising a . . . ” does not exclude additional same element in the process, method, article or device comprising the element.

The monitoring device, the monitoring system and the method for acquiring a state parameter according to the present disclosure have been described in detail above. The principle and the embodiments of the present disclosure are described herein with reference to the particular examples, and the description of the above embodiments is merely intended to facilitate to understand the method according to the present disclosure and its core concept. Moreover, for a person skilled in the art, according to the concept of the present disclosure, the particular embodiments and the range of application may be varied. In conclusion, the contents of the description should not be understood as limiting the present disclosure.

1. A monitoring device, wherein the monitoring device comprises: a sensor interface, a controller, a switch element and a monitoring element;
the sensor interface is electrically connected to the monitoring element and the switch element, and the sensor interface is configured to be accessed by a sensor;
the controller is connected to the sensor interface via the monitoring element and the switch element that are connected in parallel;
the monitoring element is configured to transmit a monitoring information to the controller when the sensor interface is monitored to be accessed by a sensor; and
the controller is configured to, according to the monitoring information sent by the monitoring element, control an on-off state of a switch in the switch element, to select a specified sensor to be powered on among the accessed sensors, and obtain a sensing information of the specified sensor.

2. The monitoring device according to claim 1, wherein the controller is further configured to, in an operating process of the specified sensor, receive a current or voltage signal outputted by the specified sensor, and according to the current or voltage signal and a physical-parameter conversion formula corresponding to the specified sensor, obtain a state parameter corresponding to the sensing information by calculation.

3. The monitoring device according to claim 2, wherein the monitoring device further comprises: a communication element;
the communication element is connected to the controller;
the controller is further configured to output the state parameter to the communication element; and
the communication element is configured to receive the state parameter sent by the controller, and send the state parameter to a receiving device.

4. The monitoring device according to claim 3, wherein the communication element is any one of a wireless-fidelity module, a Bluetooth module, a fourth-generation-mobile-communication-technology module and a fifth-generation-mobile-communication-system module.

5. The monitoring device according to claim 1, wherein the monitoring device further comprises: a power-supply interface; and
the power-supply interface is electrically connected to a pin of the sensor interface, and the power-supply interface is configured to be connected to a power supply, to provide electric energy to operation of the monitoring device.

6. The monitoring device according to claim 5, wherein the monitoring element is a current monitoring chip; and
the current monitoring chip is configured to, when the sensor interface is accessed by the sensor and the power-supply interface switches on the power supply, transmit a current signal flowing through the current monitoring chip to the controller.

7. The monitoring device according to claim 6, wherein the controller is further configured to, according to a current signal sent by the current monitoring chip, identify an accessing state of the sensor interface.

8. The monitoring device according to claim 3, wherein the monitoring device further comprises: a locating element; and
the locating element is connected to the communication element, and the locating element is configured to locate a position information of the monitoring device, and send the position information to the receiving device.

9. The monitoring device according to claim 1, wherein the quantity of switches comprised in the sensor interface is equal to the quantity of switches comprised in the switch element, and each of the sensor interfaces is electrically connected with one of the switches respectively.

10. The monitoring device according to claim 1, wherein each of the sensor interfaces corresponds to one type of sensor.

11. The monitoring device according to claim 1, wherein the sensing information comprises a gas-flow-state information.

12. A monitoring system, wherein the monitoring system comprises: a breathing device, a sensor and the monitoring device according to claim 1.

13. The monitoring system according to claim 12, wherein the sensor accesses the sensor interface of the monitoring device, and the monitoring device is provided inside of a gas-flow channel of the breathing device.

14. The monitoring system according to claim 12, wherein the monitoring system further comprises: a receiving device; and
the receiving device is configured to receive state parameter inside of a gas-flow channel of the breathing device that is sent by the monitoring device, and the position information sent by the monitoring device.

15. The monitoring system according to claim 14, wherein the receiving device is provided with an alarming element; and
the alarming element is configured to, when the state parameter triggers a preset alarming condition, generate an alarming information, and send the alarming information to a target device.

16. The monitoring system according to claim 12, wherein the sensor comprises:
one or more of a pressure sensor, a flow sensor, a temperature sensor, a humidity sensor, an oxygen-concentration sensor, a position sensor and a carbon-dioxide-concentration sensor.

17. A method for acquiring a state parameter, wherein the method is applied to the monitoring system according to claim 12, and the method comprises:
determining whether the sensor interface is accessed by a sensor of a specified type according to a received current signal of the monitoring element;
controlling, by the controller, a switch in the switch element corresponds to the sensor interface to be conductive, when the sensor of the specified type is determined to be accessed to the sensor interface; and
acquiring, by the sensor of the specified type, a state parameter inside of a gas-flow channel of the breathing device.

18. The method according to claim 17, wherein after the step of acquiring, by the sensor of the specified type, the state parameter inside of the gas-flow channel of the breathing device, the method further comprises:
sending the state parameter to a receiving device; and
generating, by the receiving device, an alarming information, when the state parameter triggers a preset alarming condition, and sending, by the receiving device, the alarming information to a target device.

19. A calculating and processing device, wherein the calculating and processing device comprises:
a memory storing a computer-readable code; and
one or more processors, wherein when the computer-readable code is executed by the one or more processors, the calculating and processing device implements the method for acquiring a state parameter according to claim 17.

20. (canceled)

21. A computer-readable medium, wherein the computer-readable medium stores computer-readable code, when the computer-readable code is executed by one or more processors, the processor implements the method for acquiring a state parameter according to claim 17.
